(19) United States
(12) Patent Application Publication
(10) Pub. No.: US 2014/0088529 A1
(43) Pub. Date: Mar. 27, 2014

(54) Minimally Invasive Endoscopic/Laparoscopic Highly Absorbent Surgical Devices, Methods and System

(71) Applicant: Bradley P. Bengtson, Grand Rapids, MI (US)

(72) Inventor: Bradley P. Bengtson, Grand Rapids, MI (US)

(21) Appl. No.: 14/040,022

(22) Filed: Sep. 27, 2013

Related U.S. Application Data
(60) Provisional application No. 61/706,393, filed on Sep. 27, 2012.

(51) Int. Cl. A61F 13/36

(52) U.S. Cl. CPC A61F 13/36
USPC 604/360; 604/385.01

(57) ABSTRACT

Devices, systems and methods related to fluid control during laparoscopic surgical methods are disclosed. A device includes a highly absorbent surgical sponge device comprising highly absorbent material capable of being inserted through a port or cannula, which has been inserted into a body cavity. The device may include a fluid conduit coupled to the absorbent pad to remove fluids from or introduce fluids into the body cavity through the device during the course of surgery.

### RELATED APPLICATIONS

This application claims the benefit of co-pending U.S. Provisional Patent Application Ser. No. 61/706,393, filed Sep. 27, 2012, and entitled “Endoscopic/Laparoscopic Absorbent Devices, Methods and System,” which is incorporated herein by reference.

### BACKGROUND OF THE INVENTION

Currently, up to 30% of general surgical, and many subsurgical, procedures employ an endoscope. All current open procedures utilize sponges of some sort to facilitate the surgical procedure. Currently there is no sponge available for intraoperative minimally invasive or laparoscopic assisted procedures. The present invention relates to the design and use of a family of novel, intraoperative, highly absorbent surgical sponge devices, and, more particularly, to the design and use of surgical sponge devices utilized in minimally invasive or laparoscopic surgical procedures, including the specific unique new methods and accompanying systems.

Minimally invasive surgery, including laparoscopic surgery, endo-assisted procedures and other similar procedures involve surgical procedures performed through small incisions where cannulas are placed through the skin into an internal cavity or space such as the abdominal cavity, chest or a joint space, allowing for small, fiber optic cameras and laparoscopic specific instrumentation enabling surgeons to perform surgery inside a body cavity such as the abdomen, chest or extremities. Such surgeries may represent up to 30% or more of all procedures in some institutions and are growing in frequency. Generally, the surgery involves making a small incision or a series of small incisions into the skin allowing access to a body cavity or joint or other internal body space which may then be inflated with a gas, such as air or carbon dioxide. Small introduction tubes, called cannulas are inserted with the help of a trocar through the incision into the inflated abdomen. The pathway formed by the cannula allows the insertion of instruments, cameras, and/or other surgical devices through the incision, thereby minimizing tissue trauma, scarring from surgery, decreasing pain following the procedures, and reducing recovery time.

As with other surgical procedures, the removal of bodily fluids, such as irrigation fluid introduced by the surgeon, blood, exudates and all blood products during the procedure is a concern for minimally invasive surgery as it is for open surgery with any of these blood or body fluids decreasing visualization for the surgeon, which interferes with the operation. Gauze and sponges are normally used to remove or absorb bodily fluids during open surgical procedures. However, such materials are not necessarily conducive for use in minimally invasive surgery, as the requirement of inserting the material through the cannula is not the easiest task to perform. In addition the current sponges are not very absorbent, can fragment and also carry a great deal of lint and surgical debris into the body. There is currently no endoscopic sponge device utilized in surgery and thus this embodiment describes a family of brand new devices, methods and systems that will revolutionize minimally invasive surgery and allow surgeons the benefit of sponge use both inside and outside the body. Raytec® or other sponge products, or Kittner dissectors may be placed through a cannula, but are not typically used or have significant limitations because of multiple issues described above. These products fragment, leave debris, are difficult to remove, and do not transmit any fluid or allow suction of blood or fluid through them.

Prior art materials, such as Kittner gauze material, are occasionally used during laparoscopic surgeries. This Kittner device is really more of a dissector and does not actually absorb blood or fluid. Other materials, which include devices commonly referred to as the “cigarette sponge”, may be inserted through a cannula to be used as a sponge in an internal surgical site. These sponges are incapable of fluid absorption and must be replaced frequently, and again may fragment. In addition they tend to adhere or stick to open surgical wounds or dissection sites and then may cause additional bleeding when removed verses our new device which is absorbent but also non-adherent. These other devices are essentially a rolled up cotton sponges, and although they may be helpful to do blunt dissection, they are inadequate to do all that is required for fluid transduction and removal, and are more commonly used to dissect tissues verses a true sponge utilized in an open procedure.

### SUMMARY OF THE INVENTION

The present invention is directed towards a novel endoscopic surgical device and related materials, methods and systems used for minimally invasive/laparoscopic procedures. The invention is directed towards a device that comprises a highly absorbent material that can be used internally during a laparoscopic procedure to effectively remove fluids during the procedure and in contrast to current sponges that are removed prior to or alternatingly with performing laparo- or endoscopic surgical procedures on in vivo tissue, embodiments according to the present invention may be left in position and suctioned through or irrigated through and left in position during the procedure.

The present invention is also directed towards methods of using the absorbent device during endoscopic and laparoscopic procedures. The device may also be facilitated and introduced with a disposable sleeve, port system and also insertion device, which may be specifically fitted and contoured to the new device/apparatus and may also be packaged with a blunt, grasping endoscopic forceps that will not fragment the sponge device, thereby facilitating placement in the body cavity and also removal. Also, the sponge may be placed within one or more absorbable or non-absorbable sleeve(s) that may facilitate the introduction of the sponge-device into the body cavity through the port. An additional lubricant or slippery coating may be placed on the outside and/or inside of the introducing sleeve to facilitate placement through the cannula.

The highly absorbent sponge device can also include tubing, such as a perforated silicone type tubing that could be placed inside the sponge device and placed on suction through one of the fluid ports for the removal of blood, body fluids or irrigation fluid during a laparoscopic procedure, as well as for the delivery of medicinal materials during a laparoscopic procedure. The sponge medical device attached to the silicone or plastic like tubing may also be left inside the body for a short time and used as a drain.

The absorbent sponge device may be impregnated with a variety of materials, pharmacologic agents, antiseptics, antibiotics, or other fluids and particles to facilitate, direct or increase the function of the absorbent device. This would further include pharmaceuticals, antimicrobials, antiseptics, anticoagulants, coagulants, hydrophobic or hydrophilic materials to make it more or less absorbent of fluids and other drugs and pharmaceuticals.

It appears the present device also has coagulation properties that actually facilitate the clotting and coagulation of blood vessels and also helps provide a surface to directly initiate the coagulation cascade, and collect clotting factors, blood and platelets that may also initiate clotting.

In addition a suction device may be incorporated or placed on or within the absorbent device and will transmit and clear fluid from the device itself, thus allowing for a functional working end and a suction end that can be run or suctioned by an assistant or surgeon, or simply connected passively to an outside or internal suction source allowing for continuous clearing of fluid, blood and blood products throughout the procedure thus allowing better visualization during surgery. This novel concept has not been defined or implemented in surgery because an absorbable transmitting sponge device has not been discovered or implemented to have draw-through, fluid clearing properties.

### BRIEF DESCRIPTION OF THE DRAWINGS

FIG. 1 is a schematic view of an assembly including a trocar and cannula used during a laparoscopic or minimally invasive procedure.

FIG. 2 is a picture of an embodiment of an absorbent device according to the present invention.

FIG. 3A demonstrates the device of FIG. 2 being secured for insertion into the assembly of FIG. 1.

FIG. 3B demonstrates the device of FIG. 2 being secured for insertion into the assembly of FIG. 1.

FIG. 4A demonstrates the device of FIG. 3A being further configured for insertion into the cannula of FIG. 1.

FIG. 4B depicts a first cartridge embodiment of an absorbent device according to the present invention.

FIG. 4C depicts a second cartridge embodiment of an absorbent device according to the present invention.

FIG. 5 demonstrates the assembly of FIG. 1 being inserted into a body.

FIG. 6 demonstrates the assembly of FIG. 5 after being inserted into a body and extending into the body cavity.

FIG. 7 demonstrates the device of FIG. 4A being inserted into the cannula of FIG. 6.

FIG. 8 demonstrates the device of FIG. 4A being further inserted through the cannula and into the body cavity.

FIG. 9 shows the device of FIG. 4A being expanded and positioned within the body cavity.

FIG. 10 demonstrates the device of FIG. 4A being removed from the body cavity through the cannula.

FIG. 11 depicts an embodiment of a sponge introducer according to the present invention, including a sponge cartridge unit.

FIG. 12 shows the sponge introducer of FIG. 11 being used to expel the sponge cartridge unit.

FIG. 13A illustrates a sponge device according to the present invention having been introduced into a body cavity through a cannula.

FIG. 13B illustrates a stress riser formed on a tracer thread according to the present invention.

FIG. 14 illustrates a laparoscopic instrument being introduced through a cannula to perform a surgical operation while the sponge device of FIG. 13 remains in the body cavity.

FIG. 15 illustrates the sponge device of FIG. 13 being arranged to be removed from the body cavity after performance of the laparoscopic surgical operation of FIG. 14.

FIG. 16 shows the device being squeezed to remove excess fluids.

FIG. 17 shows the device being put into a sterilizing solution so that the device may be used again.

FIG. 18 is a perspective view of another embodiment of the present invention including an attached tubing set for the removal of fluids.

FIG. 19 demonstrates the embodiment of FIG. 28 being used to remove fluids from a body cavity.

FIG. 20 demonstrates the embodiment of FIG. 18 delivering a solution to a body cavity.

FIG. 21 depicts an embodiment of a sponge device according to the present invention, further including a conduit nipple.

FIG. 22 depicts an embodiment of a sponge device according to the present invention, further including a conduit port.

FIG. 23 demonstrates another possible feature of the absorbent device, wherein the device has a ring to allow grasping of the device.

FIG. 24 depicts the device of FIG. 23 being gripped by an insertion device.

FIG. 25 depicts the device of FIG. 24 being rotated for insertion into a body cavity.

FIG. 26 and FIG. 27 depict the device of FIG. 25 being inserted into a body cavity.

FIG. 28 depicts the device of FIG. 27 after insertion into a body cavity, with the insertion device being removed from the body cavity.

FIG. 29 depicts a device according to the present invention incorporating a Radio Frequency Identification (RFID) marker into the device.

FIG. 30 depicts a device according to the present invention incorporating an x-ray string within the device.

FIGS. 31-33 depicts the device of the present invention being used during a laparoscopic cholecystectomy/gall bladder procedure. This device could also be used to minimize bleeding of such organs as the liver and spleen, for example.

FIG. 34 illustrates a first embodiment of a sponge kit according to the present invention.

FIG. 35 illustrates a second embodiment of a sponge kit according to the present invention.

FIG. 36 illustrates a third embodiment of a sponge kit according to the present invention.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims, which may include devices of multiple types of materials such as polyvinyl alcohol (PVA) or other such polymers as well as future absorbable materials that could even be left inside the body cavity to for instance control bleeding, and may be of all types of shapes and sizes for individual applications or designed uses.

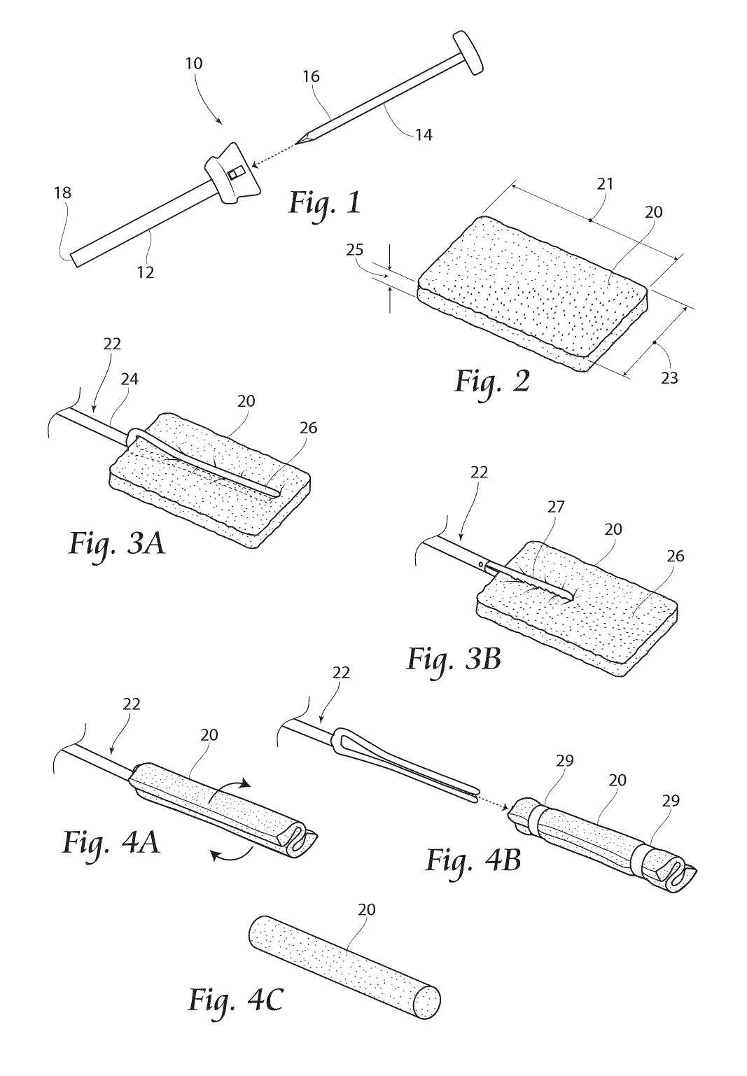

FIG. 1 is an exploded view of an assembly 10 for use during a laparoscopic procedure. The assembly 10 generally comprises a cannula 12 and a trocar 14, with the trocar 14 being insertable into the cannula 12. The assembly 10 allows for access into a body cavity to carry out a laparoscopic procedure. The trocar 14 has a shaft 16 ending in a sharpened end that is used to pierce through body tissue and allow the assembly 10 to be inserted into the body cavity. The cannula 12 generally comprises a hollow sheath 18 for the trocar 14 to pass through. The current device is non-absorbable by an animal body but a highly absorbable device may also be created and used as a foam sponge device in the future, developed generally along the principles of the present invention, as well as future generations of an all-absorbable device.

FIG. 2 demonstrates a device 20 according to the present invention, comprising an absorbent pad, extending preferably in three Cartesian dimensions. The device 20 is designed so that it can be used internally during a minimally invasive/laparoscopic surgery. The device 20 is up to 1500% more absorbent than prior art sponges used for open procedures, and is much more durable, lint and particle free, washable and also reusable within the same surgical procedure. Various fluids can be transmitted and suctioned through the device 20 as well. Also, it should be noted that the present invention is the first known laparoscopic designed absorbent device 20 created specifically for minimally invasive surgery.

The device 20 comprises a sterilizable, liquid-absorbent, and durable material that can be economically sized and configured for use and reuse during the same laparoscopic procedure. The material is preferably dust and lint free (that is, it does not flake or shed fibers or the like similar to current cotton sponges used in laparoscopic procedures). This new material is soft, non-abrasive and does not traumatize skin or deep tissue that is often very friable from a disease process such as infection or cancer. The material possesses an enhanced liquid absorbing capacity up to 1500%, which can be a very desirable characteristic, e.g., by a capability of holding at least ten times its weight in liquid. It can be used in the body cavity, where the absorbent character of the material quickly absorbs blood, body fluids, as well as water and other aqueous liquids in an operative site, and as noted may also have inherent coagulation properties that facilitate the body's own clotting ability. The material is sized and configured to be quickly reconditioned outside the body cavity, where the material can be quickly rinsed and cleaned by immersion in an aqueous liquid (e.g., saline) preferably outside of a surgical field, and residual liquid easily released by manual wringing and rinsing in saline, and at that time be immediately available for return to and reuse in the operating field. The material is sufficiently durable so that it can be rinsed, cleaned, and wrung to release residual liquid repeatedly and returned for repeated reuse in the body cavity, over and over again during the course of a single surgical procedure. At the end of the surgical procedure, the material can be disposed of as medical waste, with much less bioburden to our world.

The material that is particularly suited for use in the manner described is a cellulose based material, such as a synthetic chamois. Such a material exhibits the above-noted desired characteristics; notably, an enhanced liquid absorbing and release/conditioning capacities, as well as the durability to withstand repeated reuse. An absorbent device 20 comprising synthetic chamois can be repeatedly used to absorb blood, body fluids, as well as water and other aqueous liquids within a body cavity, then quickly rinsed, wrung to release residual liquid, and returned for reuse in the body cavity, again and again during the course of a given surgical procedure. Such suitable materials for the absorbent device 20 include polyvinyl alcohol, polyurethane, polyvinyl acetate, polypropylene, polyester, polytetraflouroethylene (PTFE), polyglycolic acid and other like-based materials, including other esters. Other suitable materials, such as cellulose materials, micro denier or other cleaning textiles, foam like materials or fabrics such as elastomers and/or synthetic materials may also or alternatively be used. Preferably, at least a portion of each surgical sponge device 20 consists essentially of polyvinyl alcohol (PVA) sponge material. Most preferably, a majority of each surgical sponge device 20 consists essentially of polyvinyl alcohol (PVA) sponge material. The surgical sponge devices 20 may also include coatings, e.g. anti-microbial and anti-fungal coatings, or soaked in such materials like antiseptics, antimicrobials and even antibiotic solutions.

The preferred materials for the surgical non-absorbent devices 20 are highly absorbent compared to prior art sponges and pads. These new nonabsorbent devices 20 have absorbency ranges of more than 100 percent increase of the traditional surgical sponges, to a 1000 percent increase absorbency compared to traditional sponges, and over 1500% increase in absorbency over some traditional sponges on the market and utilized in surgery every day. The absorbent devices 20 also have an increased absorption rate per area, with absorption rates of at least 0.70 g/in² and up to and above 5.00 g/in². This clinically equates to placing the pad on a table and placing a large amount of water on the table top with the water immediately being soaked into the device 20.

As shown in FIG. 2, the device 20 preferably has a rectangular shape with a length 21, a width 23, and a thickness 25, but it can be of any shape, e.g. circular, triangular, and is designed of material so that it may be easily cut to any preferred shape. Indeed, as shown in FIG. 4C, the device 20 may be provided in shapes other than a generally planar sponge, such as a cylinder. As such, embodiments according to the present invention can be used with all standard diameter cannulas 12, e.g. open diameters of up to 5 millimeters (mm), 6 mm, 9 mm, 10 mm, 15 mm etc. The absorbent devices 20 may be provided in a variety of thicknesses 25, which may be related to the cannula diameter, such as about 0.2 times the cannula diameter to about 0.8 times the cannula diameter, with about 0.2 times to about 0.5 times being preferred. The other two dimensions, length 21 and width 23 at orthogonal disposition to the pad thickness 25, may be substantially larger, such as a width 23 from the thickness 25 up to about sixty times the thickness, and such as a length 21, equal to or greater than the width 23, and up to about eighty times the thickness 25. If the device 20 is provided in a cylindrical shape, the diameter of such cylindrical sponge may be up to and including the diameter of the cannula 12. The device 20 can also be considered an absorbent device 20, as noted above. The device 20 may also be coated or impregnated with one or more materials such as silver, an antiseptic, an antibiotic, a haemostatic agent (e.g. a coagulant), an anesthetic (e.g. lidocaine), a vasoconstrictor (e.g. epinephrine), or other pharmaceuticals or preparations. Additionally or alternatively, absorbent sponge devices 20 according to the present invention, of one or more sizes, may be provided for use outside, yet in contact with a body, such as a substitute for standard wound dressings, like gauze.

FIG. 3A shows the device 20 being coupled to an insertion device 22 that will allow the absorbent device 20 to be eventually inserted into a body cavity. The insertion device 22 preferably has an elongated shaft 24 with a gripping end 26 for grasping the absorbent device 20. For example, the gripping end 26 could comprise a pair of retractable tongs that will securely grasp the absorbent device 20. Other designs such as a claw-like arrangement or a fork-like design for piercing the device 20 could also be used. As shown in FIG. 3B, the insertion device 22 may have atraumatic ribbed teeth 27 that are similarly used in other medical devices, such as those used for bowel or intestine retraction. The insertion device 22 is preferably made of a plastic, silicone, or other biocompatible polymer materials that may also be coated with a lubricant to facilitate insertion and removal without affecting the functionality of the sponge device 20.

Referring to FIG. 4A, the insertion device 22 may be rotated to condense the absorbent device 20 into a more compact arrangement for insertion into a body cavity. The absorbent device 20 is formed into a general cylindrical shape so that it can eventually be inserted the assembly 10, as will be described below.

Additionally or alternatively, the device 20 may be formed into a multi-piece sponge cartridge unit 20′ as shown in FIG. 4B. This embodiment of a sponge cartridge unit 20′ includes the sponge device 20 and one or more cartridge bands 29. The bands 29 are suited to maintain the device 20 in a preferred configuration, such as rolled or folded, prior to and during insertion into a body cavity. The bands 29 may be made from any desirable material, and may be water soluble or not. If the bands 29 are not water soluble, they are preferably permanently attached to the device 20 so that they may be removed with the device upon extraction from the body cavity. Water soluble bands may be formed from any non-toxic water soluble substance but are preferably formed at least partially from a water soluble polyvinyl alcohol material, such as that disclosed in U.S. Pat. No. 3,892,905, incorporated herein by reference in its entirety.

FIG. 4C provides an alternate embodiment 20″ of a sponge cartridge unit, including a unitary sponge device 20 of generally cylindrical shape. Any of the cartridge units 20′,20″ may be used alone or in conjunction with an introducer device, such as that shown in FIGS. 11 and 12, and described below.

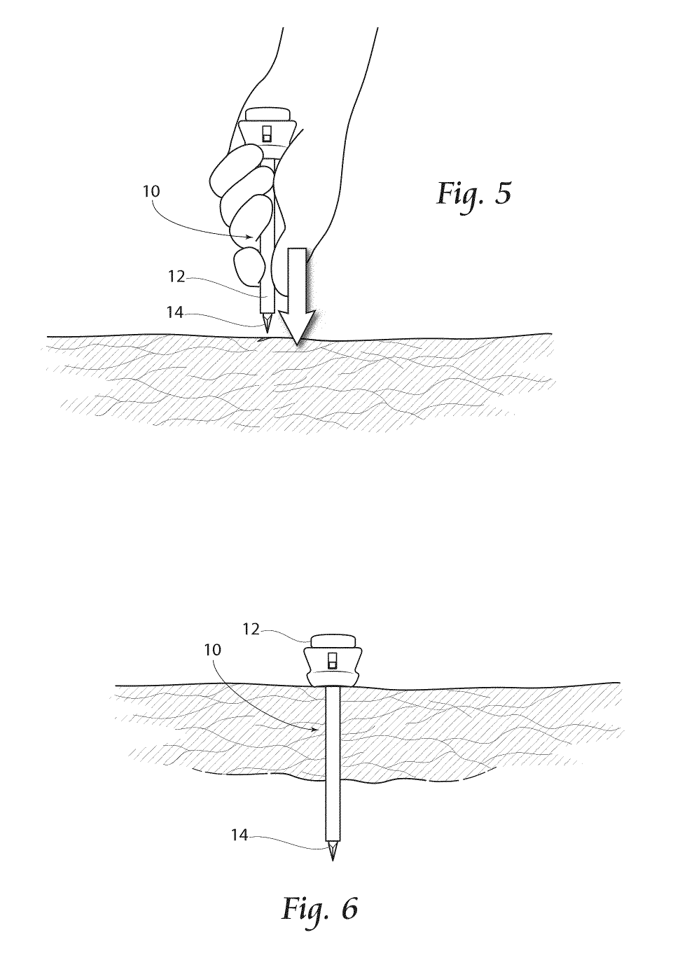

Referring now to FIGS. 5 and 6, the assembly 10 is shown being inserted into tissue and into a body cavity. The sharpened end of the trocar 14 is pushed downwardly into the tissue and eventually extending into the body cavity, as shown in FIG. 6. Once inserted into the body cavity to a desired depth, the trocar 14 is removed from the assembly 10, with the cannula 12 remaining in place to allow a pathway into the body cavity.

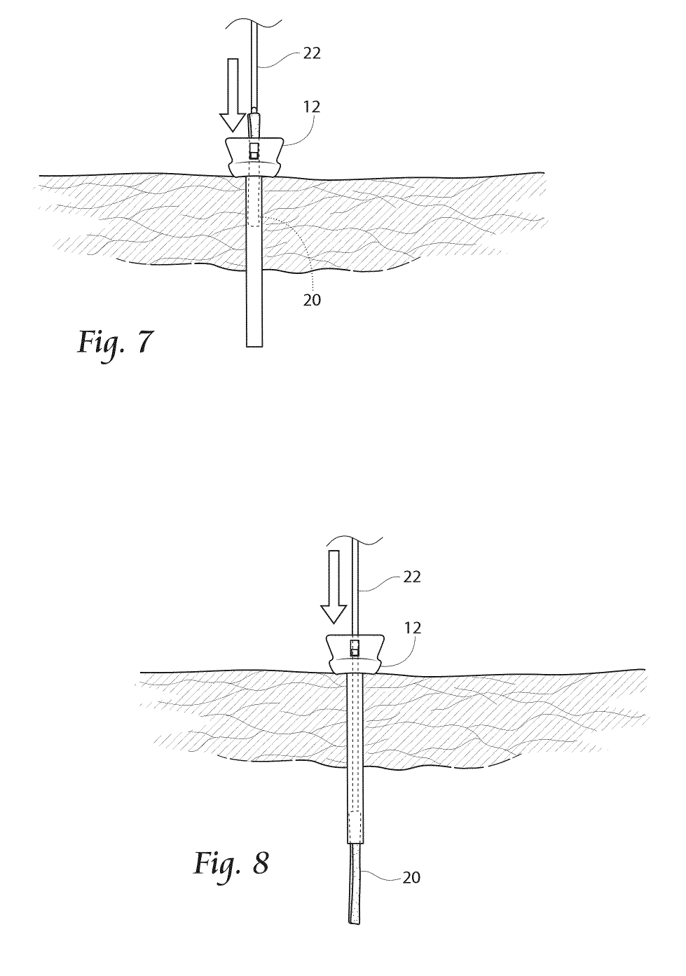

As shown in FIG. 7, the absorbent device 20 is being inserted into the cannula 12 by way of the insertion device 22, as described above. The absorbent device 20 is in the compact, cylindrical position, which allows it to pass through the cannula 12, into the body cavity, as depicted in FIG. 8. Multiple devices 20 may be inserted in series if desired.

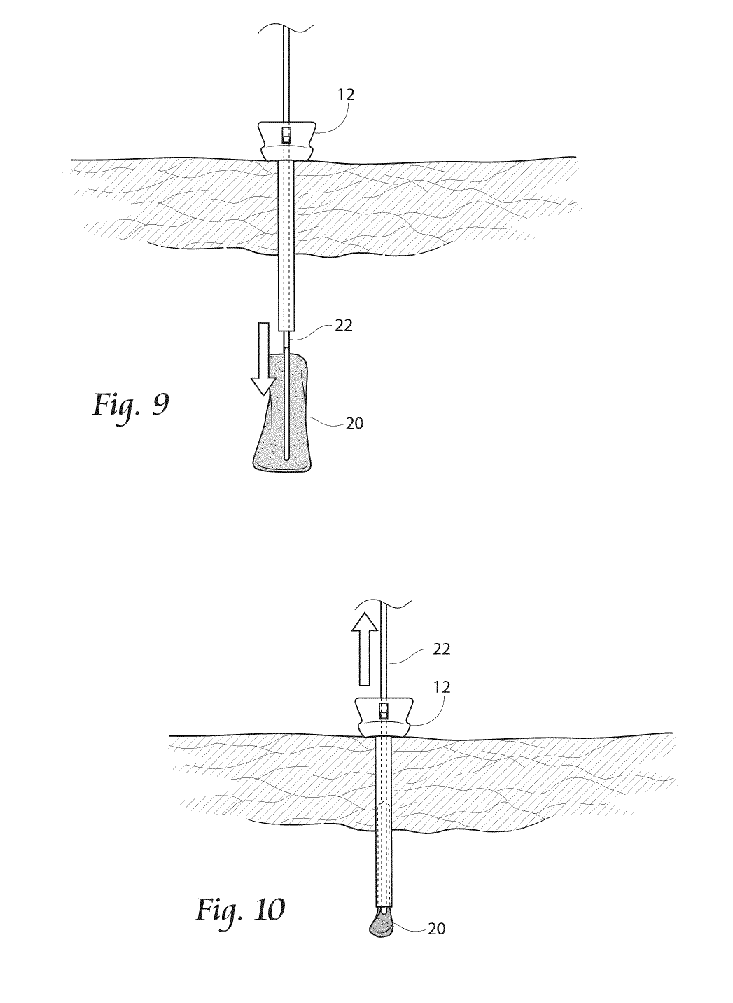

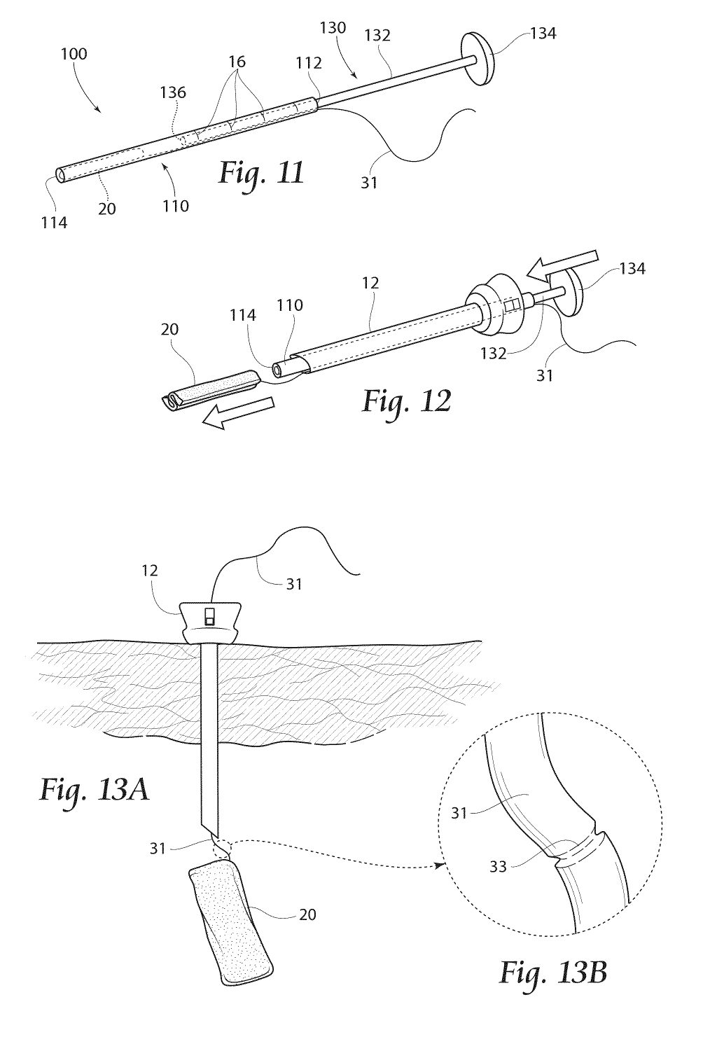

Once in position, the absorbent device 20 can be expanded into its general shape that it had prior to being inserted, as depicted in FIG. 9. The insertion device 22 may be removed from the absorbent device 20, or may remain attached to the absorbent device 20. The absorbent device 20 can remain in position until it has become sufficiently saturated with fluids and needs to be removed, or simply having fluid suctioned away while in place, removing fluid, blood and blood products. As stated above, the absorbent device 20 is capable of absorbing up to 1500% more fluids than prior art sponges and materials, which reduces the number of times that the absorbent device 20 will need to be removed from the body cavity, and easily transduce fluid and blood and simply suctioned clear of the operating field. As shown in FIG. 10, once the absorbent device 20 is to be removed, the insertion device 22 may again be employed to remove the absorbent device 20 through the cannula 12.

FIGS. 11 and 12 provide an embodiment of a sponge introducer 100 according to the present invention. The introducer 100 includes an applicator tube 110 and a tamper 130. The applicator tube 110 is preferably a cylindrical tube extending between a first open end 112 and a second open end 114. Disposed along the length of the applicator tube 110 is preferably one or more depth indicator marks 116, which may be equally spaced from one another, if a plurality of marks 116 are provided. The tamper 130 preferably includes a tamping rod 132 connected to a handle 134. The tamping rod 132 includes a free end 136, which may be inserted into the first end 112 of the applicator tube 110. When the tamping rod 132 is inserted completely into the applicator tube 110, a portion of the rod 132 preferably protrudes from the second end 114 of the tube 110.

Referring also to FIG. 13, in addition to FIGS. 11 and 12, the use of the introducer 100 may be described. To use the sponge introducer 100, a sponge device 20 may be positioned within the applicator tube 110. The sponge device 20 may be a rolled device or a sponge cartridge unit, as previously described in connection with FIGS. 4A-4C. The device 20 may include a tracer thread 31 extending therefrom. The tamper 130 is placed into the first end 112 of the applicator tube 110, and the tube 110 is placed inside of a cannula 12, to a desired depth, which may be indicated by the provided depth marks 116 on the tube 110. Once the tube 110 is in a desired position with respect to the cannula 12, the tamper 130 may be used to push the sponge 20 out of the tube 110 and into the body cavity. The tracer thread 31 may extend through the cannula 12 and be displayed outside the body as a reminder to the surgeon that there remains a sponge 20 within the body cavity. The tracer thread 31 is thus used to help ensure that the surgeon will return to remove the sponge 20 at the end of the operation. The tracer thread 31 may be provided with the sponge 20, or may be temporarily placed by the user, e.g. surgeon or assistant, of the device. If the tracer thread 31 is provided with the sponge device 20 right out of the package, the tracer thread 31 preferably includes one or more stress risers 33 along its length. The purpose of the stress riser 33 is to hamper the ability of a user to pull on the tracer thread 31 in an attempt to retract the sponge 20 from the body cavity after placement. The stress riser 33 is designed to fail at an applied tension force on the thread 31 that is less than a tension force that would be required to tear the sponge device 20 at the location at which the thread 31 is attached to the device 20. In this fashion, if the thread 31 is attempted to be used to extract the sponge 20 from the body cavity, the thread 31 would break before the sponge 20 would rip, thereby minimizing sponge shrapnel being disposed in the body cavity.

During the use of an applicator tube 110, a lubricant may be utilized to ease delivery of the sponge 20. Lubricant may be provided to be applied to the sponge 20 and/or applicator tube 110 to decrease friction therebetween. Lubricant may additionally or alternatively be provided to be applied to the applicator tube 110 and/or cannula 12 to reduce friction therebetween.

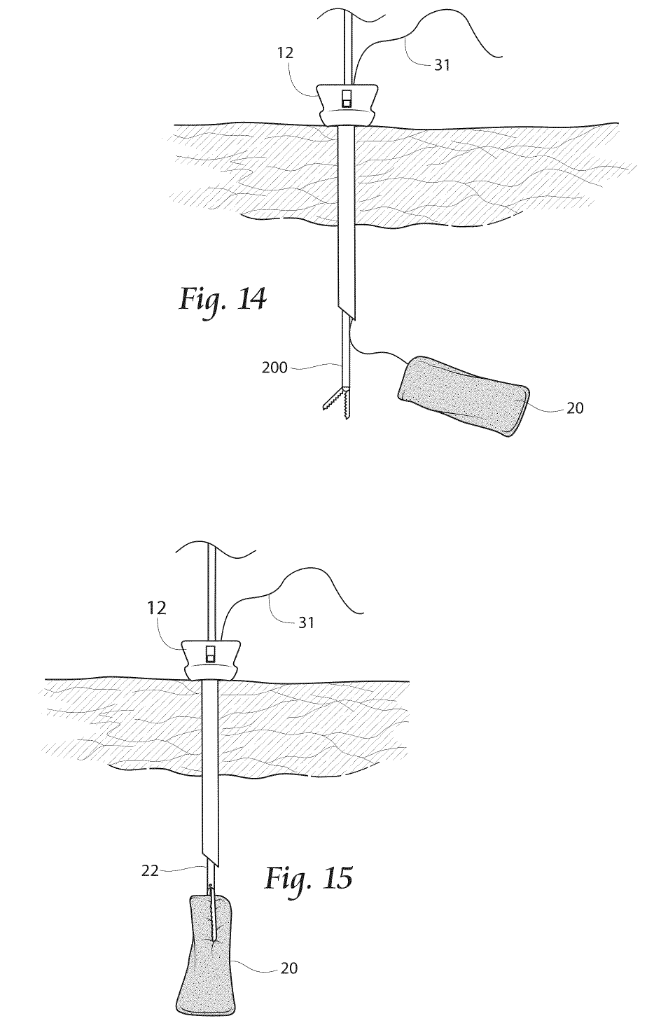

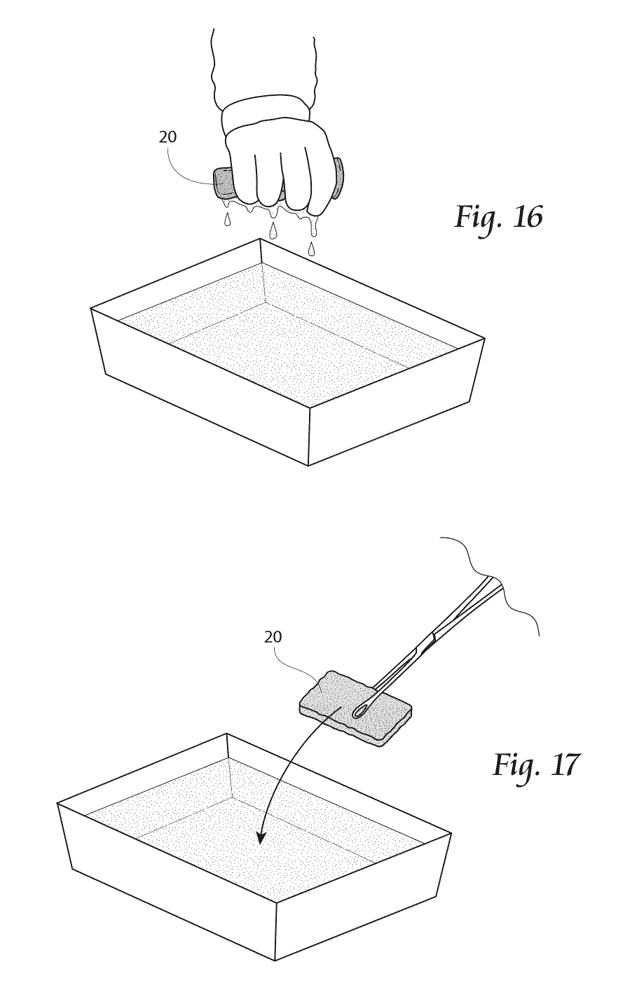

As can be seen in FIG. 14, the sponge device 20, which may have been placed in the body cavity through the cannula 12 using the introducer 100, may be left in place during a laparoscopic surgical procedure performed using a laparoscopic instrument 200. Additionally or alternatively, the sponge 20 may be left in the body cavity for a predetermined amount of time or until the sponge reaches a desired saturation point.

When it is desired to remove the sponge 20 from the body cavity, a gripping device 22 as previously described, may be inserted into the cannula 12 and into the body cavity, to grasp the sponge 20 and retract it through the cannula 12 and out of the body to be discarded or cleaned for reuse. Alternatively, the laparoscopic surgical instrument 200 could be used to retrieve the sponge 20.

Once out of the body, the absorbent device 20 may be wrung out of the bodily or irrigation fluids absorbed in the body cavity, as depicted in FIG. 16.

The design and the material of the absorbent device 20 allows the absorbent device 20 to be reused, if necessary, thus reducing the overall number of absorbent devices 20 being used during a single procedure compared to prior art materials and procedures. As shown in FIG. 17, the absorbent device 20 may be placed into a sterilization solution, which will allow the absorbent device 20 to be cleaned and allow it to be reinserted into a body cavity, if desired.

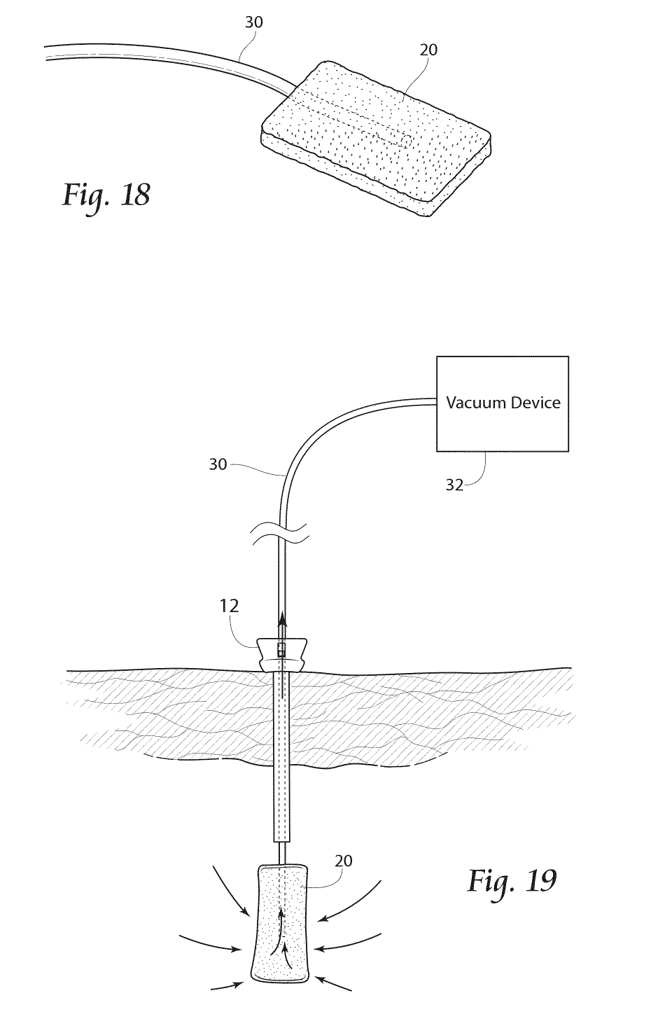

The present invention also provides advantages over prior art devices in that the absorbent device 20 is capable of actively remove or deliver fluids during a laparoscopic procedure. For example, the absorbent device 20 as shown in FIG. 18 has a tubing 30 coupled to the absorbent device 20. The tubing 30 establishes a fluid conduit between the device and an external device. The tubing 30 may have a perforated end that will allow the tubing 30 to be slid into or attached to the absorbent device 20. The tubing 30 may be embedded in the absorbent device 20, or may be attached by external means, such as sewing, adhesive, or other means for securing the tubing 30 to the absorbent device 20. The absorbent device 20 can be inserted into the body cavity, as shown in FIG. 19. Once the absorbent device 20 is in the desired position, the tubing 30, which may be connected to an external suction device 32, can actively remove fluids from the body cavity, thereby allowing the absorbent device 20 to remain in place without the need to rinse or wring out the device during a laparoscopic procedure. Such an arrangement minimizes the need to remove the absorbent device 20 during a procedure, or possible allows for the absorbent device 20 to stay in place during an entire procedure, and removed upon completion of the substantive procedure, prior to removal of the cannula 12. Additionally or alternatively, the device 20 may be left in position inside a body cavity to function as an advanced drain connected to any form of suction or negative pressure as to drain fluid out of an internal closed space (without or after the use of a cannula 12) effectively draining fluid, blood, blood products, exudates that may occur following surgery or trauma. The device 20 could then be removed a future date, such as by open incision, or through a later-placed cannula.

As shown in FIG. 20, the absorbent device 20 may also be used to deliver medicines or other fluids that may be useful in aspirating the body cavity. For example, the tubing 30 may be used to transfer coagulants to the body cavity or vasoconstrictors, e.g. epinephrine, to stop bleeding in the cavity. The absorbent device 20 of the present invention thereby provides a unique device 20, which not only may comprise materials that have haemostatic qualities, e.g. blood clotting, coagulation properties, it may also be designed to deliver materials and medicines to the surgical site, as well as growth factors or other new products that accelerate or improve wound healing.

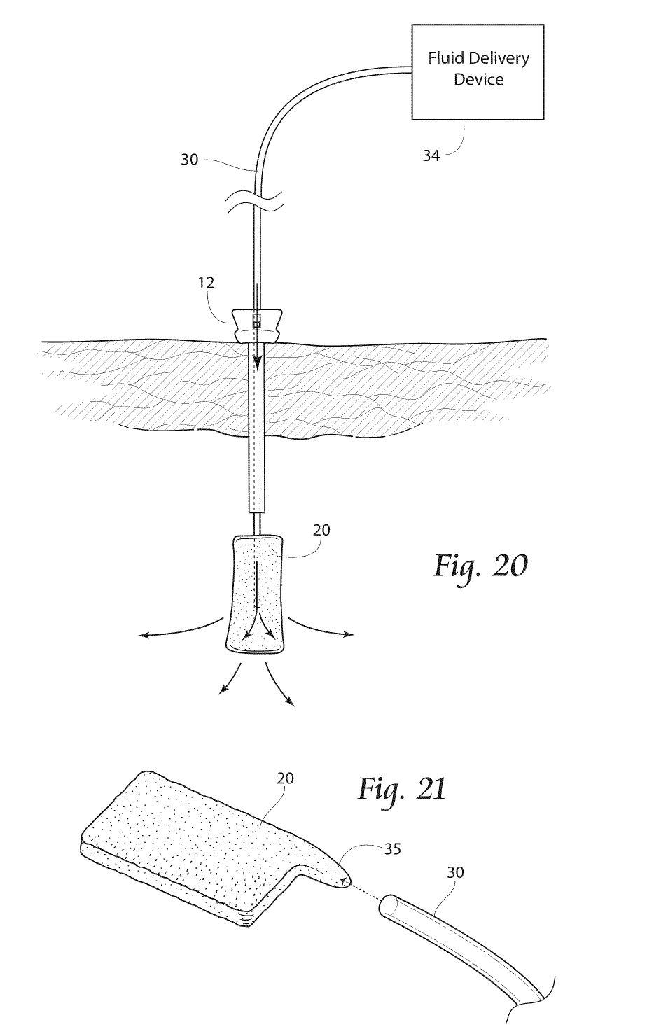

To enable efficient coupling to fluid suction or delivery tubing 30, the sponge 20 may be provided with a tubing interface. A first tubing interface is shown in FIG. 21. The interface is provided as a protrusion, such as a nipple 35, which may be formed contiguously with the rest of the sponge 20. The tubing 30 is interfaced with the nipple 35 by the nipple 35 being inserted into the tubing 30, which may remain frictionally engaged therewith, or be adhered thereto. A second tubing interface is shown in FIG. 22. This interface is a tubing port 37, preferably formed from the same material as, and contiguously with, the rest of the sponge 20. The tubing 30 may be coupled to the sponge 20 by being inserted into the tubing port 37 and remaining frictionally engaged therewith, or adhered thereto.

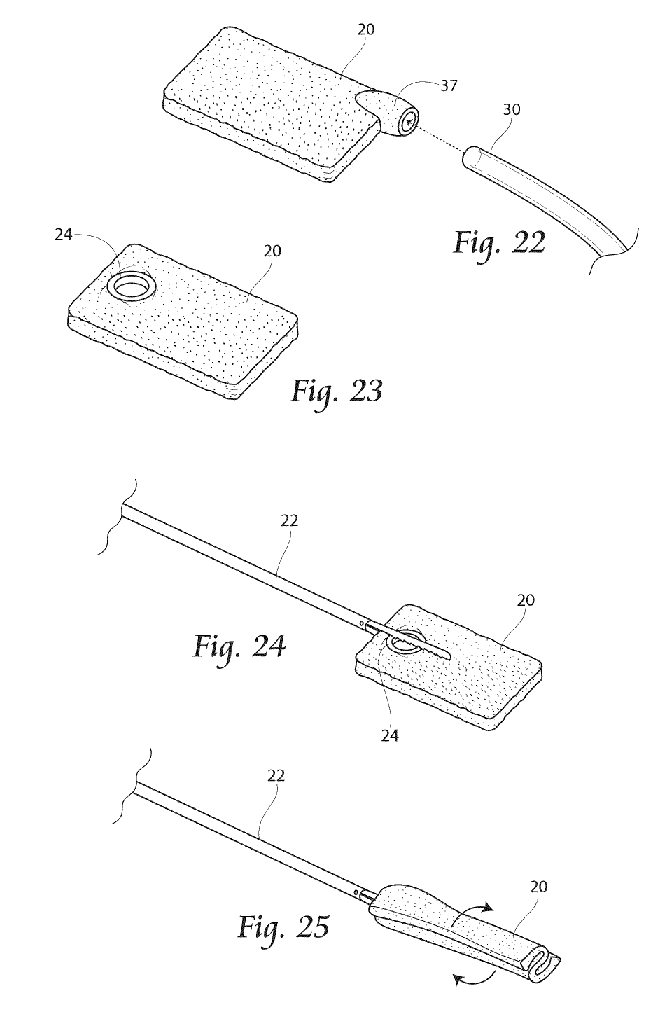

The absorbent device 20 of the present invention is designed for easy insertion and removal into a surgical cavity during minimally invasive surgery. The absorbent device 20 may further comprise other features to assist in the insertion and removal of the absorbent device 20 from the internal operating area. For example, FIG. 23 depicts the absorbent device 20 including a ring. The ring allows the absorbent device 20 to be grabbed by an external device 22, e.g. forceps, so that the absorbent device 20 can be positively grabbed and retained for insertion into the body cavity. The ring is depicted as being a grommet within the absorbent device 20, itself, but it could be designed to connect or intersect to the periphery of the absorbent device 20.

FIGS. 24 and 25 show the absorbent device 20 being gripped by a pair of forceps so that the device 20 can be reconfigured for insertion through a cannula 12 into a body cavity. As previously depicted, the ability of the absorbent device 20 to be grasped and manipulated into a different shape for insertion further minimizes any contamination associated with the device 20 being unnecessarily handled by a person.

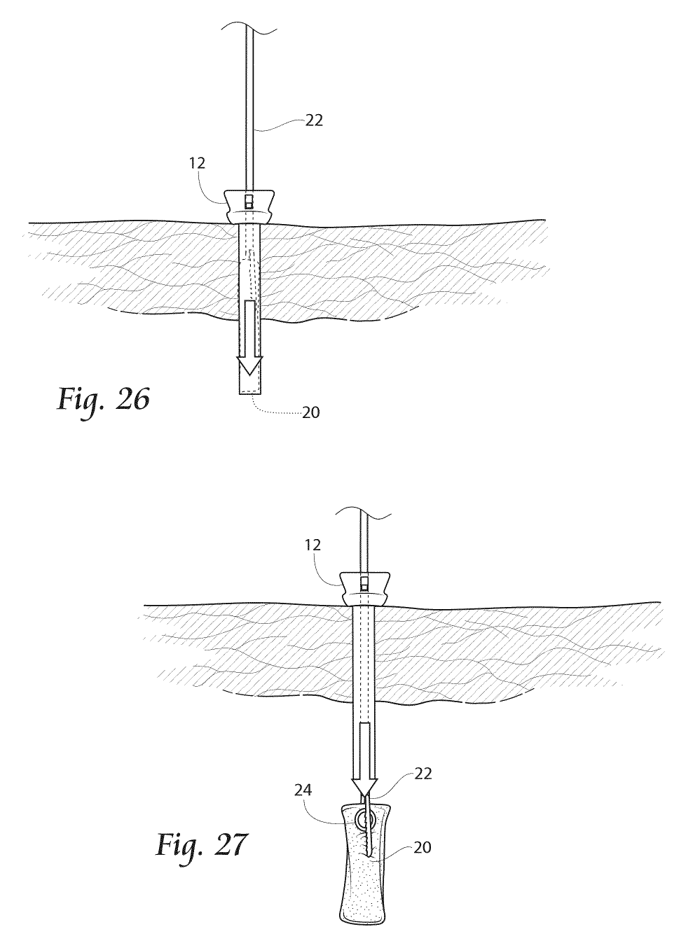

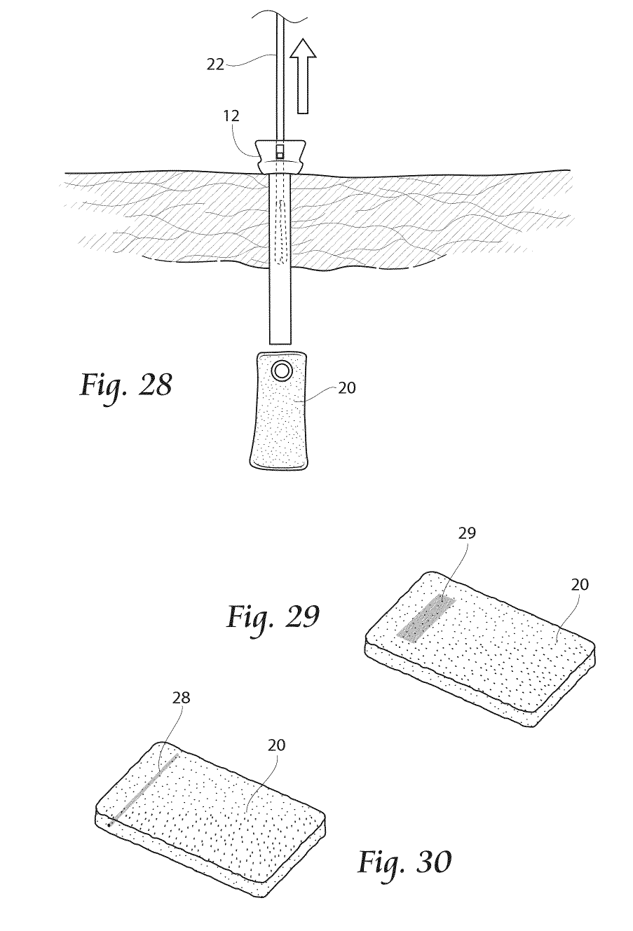

Once in position, the absorbent device 20 can be placed into a sheath 18 or tube, as previously noted, and inserted through a cannula 12, as shown in FIG. 26. After the device 20 is positioned within the internal operating cavity (FIG. 27), the forceps or other gripping device 22 can be retracted out of the operating cavity (FIG. 28), with the absorbent device 20 remaining within the internal operating cavity. Once it is necessary to remove the absorbent device 20, the ring 24 assists the reinserted forceps to positively grab the absorbent device 20 and remove the absorbent device 20.

The absorbent device 20 may also comprise other features that aid in the utility of the present invention. For example, a Radio Frequency Identification (RFID) tag 26 could be incorporated into the absorbent device 20, as shown in FIG. 29. Such a tag 26 will assist in locating the device 20, either within or outside the internal operating cavity, which is an important safety and liability feature during surgical procedures. FIG. 30 demonstrates that an X-Ray tag, e.g. string 28, can also be incorporated into the absorbent device 20 so that the absorbent device 20 can be noted and located during an X-Ray procedure. It should be understood that the arrangements of FIGS. 29 and 30 can be incorporated into any of the embodiments of the present invention.

The absorbent material of the device 20 of the present invention provides an improvement for minimally invasive surgical procedures, e.g. endo- and laparoscopic procedures, that the prior art could not address, particularly with the delivery and removal of fluids from the operating area.

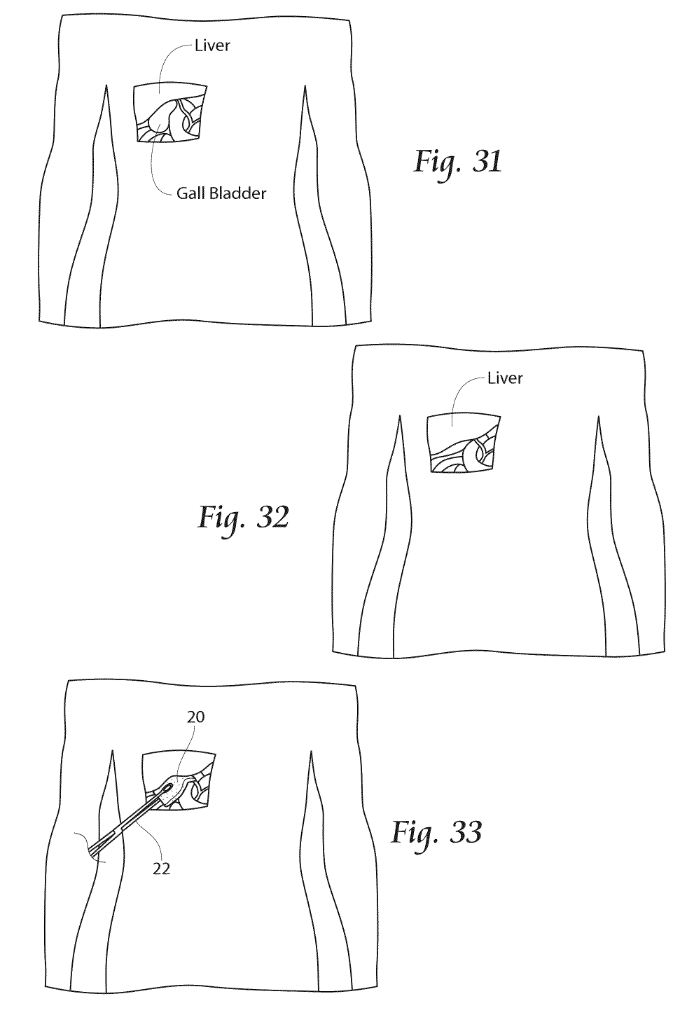

As an example, and not limiting to any type of procedure, FIGS. 31-33 depict a cholecystectomy or gall bladder operation. While such an operation will normally be performed with minimally invasive procedures, as described above, the procedure is shown with the body having a large opening to demonstrate the present invention's utility. FIG. 31 shows the liver and gall bladder prior to removal of the gall bladder. Once the gall bladder is removed, as shown in FIG. 32, the liver bed will move into the area previously filled by the gall bladder. The use of the absorbent device 20 of the present invention (FIG. 33) allows for retractment and repositioning of the liver while the procedure is being finished. The absorbent device 20 can remove fluids from the operating area, as previously described, without constantly needing to remove and replace a sponge or gauze, as with previous devices 20.

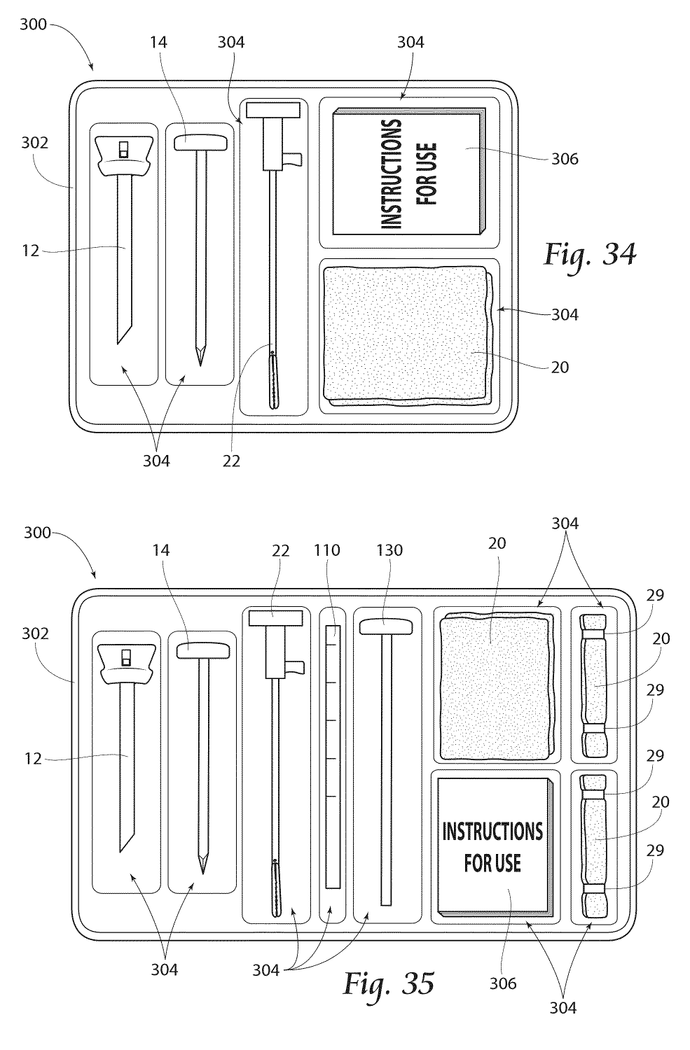

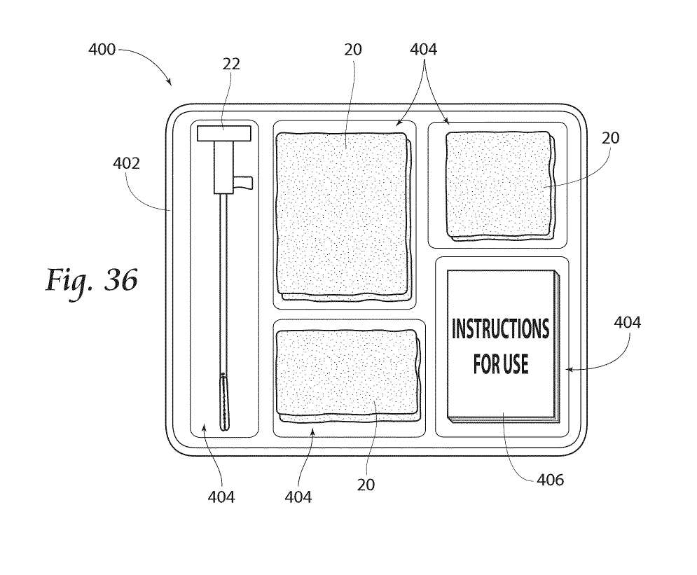

FIG. 34 depicts a first embodiment of a sponge kit 300 according to the present invention. The kit 300 preferably includes a container 302, such as a draw-formed thermoplastic tray, which may form one or more compartments 304. The compartments 304 are preferably sealable by, e.g., plastic film (not shown) which may be adhered to the tray 302. This first kit 300 includes a cannula 12, a trocar 14, a sponge retraction tool 22, one or more sponge devices 20, and instructions 306 for using the sponge 20 in conjunction with the provided system components. The instructions for use 306 generally would set forth the method steps described herein. Each provided component may be placed in its own compartment 304, or a plurality of components may share a compartment 304. Additionally, a kit 300′ may include a sponge introducer 100, as shown in FIG. 35. Additionally or alternatively, the cannula 12 and trocar 14 may be eliminated from a kit 400, and/or a plurality of sizes of sponges 20 may be provided, as shown in FIG. 36. The tray 302,402 from the kit 300,400 or a compartment 304,404 thereof, may serve as a rinsing tray during a surgical procedure, such as that shown in FIGS. 16 and 17.

The present invention not only provides improvements over prior art devices 20, but the use of the absorbent devices 20 also provides improved surgical methods and procedures, as described in herein, not previously recognized by the prior art. In addition, future developments may include a completely bodily absorbable sponge that could be used in the exact same ways but then left inside the body to provide improved healing, growth factors, coagulation etc. In addition the sponge device 20 could actually be a living tissue either human from the same or other patient or animal that may provide the same characteristics as the sponge device 20.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I claim:

1. A device to be used during a laparascopic surgical procedure, the laparascopic surgical procedure comprising a cannula inserted into a body cavity, the device comprising:
an absorbent pad capable of being inserted through the cannula and into the body cavity, the absorbent pad comprising a width, a length, and a thickness, wherein the thickness is in the range of 0.2 times an open diameter of the cannula to one times the open diameter of the cannula.

2. A device according to claim 1, wherein the absorbent pad comprises at least one material selected from the group consisting of polyvinyl alcohol, polyurethane, polyvinyl acetate, polypropylene, polyester, polytetraflouroethylene, and polyglycolic acid.

3. A device according to claim 2, wherein the absorbent pad consists essentially of polyvinyl alcohol.

4. A device according to claim 1, wherein the absorbent pad has a weight and is capable of absorbing at least ten times the weight of a liquid.

5. A device according to claim 1, wherein the absorbent pad further comprises at least one of an antiseptic, an antimicrobial, and an antibiotic.

6. A device according to claim 1, wherein the absorbent pad has a water absorption rate of at least 0.7 grams per square inch.

7. A device according to claim 6, wherein the absorbent pad has a water absorption rate of up to 5.00 grams per square inch.

8. A device according to claim 1, wherein the absorbent pad further comprises a silver coating.

9. A device according to claim 1, further comprising a grasping tool capable of receiving the absorbent pad and capable of extending through the cannula.

10. A device according to claim 1, further comprising a fluid conduit extending between a first end coupled to the absorbent pad and a second end.

11. A device according to claim 10, wherein the fluid conduit comprises a length extending through the cannula.

12. A device according to claim 11, wherein the second end is coupled to a fluid supply.

13. A device according to claim 11, wherein the second end is coupled to a vacuum supply.

14. A device according to claim 1, wherein the cannula has an open internal cannula diameter and the absorbent pad has two orthogonal dimensions greater than the open internal cannula diameter.

15. A device according to claim 1 further comprising a grommet extending through the absorbent pad.

16. A device according to claim 1, the absorbent pad further comprising an x-ray detectable material.

17. A device according to claim 1, the absorbent pad further comprising a radio frequency identification tag.

18. A method of absorbing fluid in a body cavity, the method comprising the steps of:
inserting a cannula into the body cavity; and
inserting through the cannula and into the body cavity an absorbent pad, the absorbent pad comprising at least one material selected from the group consisting of polyvinyl alcohol, polyurethane, polyvinyl acetate, polypropylene, polyester, polytetraflouroethylene, and polyglycolic acid.

19. A method according to claim 18, further comprising the step of removing the absorbent pad through the cannula.

20. A method according to claim 18, wherein a fluid conduit extends between a first end coupled to the absorbent pad and a second end.

21. A method according to claim 20, wherein after the absorbent pad is inserted into the body cavity through the cannula, the fluid conduit second end being disposed outside of the body cavity and outside of the cannula.

22. A method according to claim 21, further comprising the step of drawing a fluid from the body cavity, through the absorbent pad and the first end towards the second end.
